(19) United States
(12) Patent Application Publication
(10) Pub. No.: US 2024/0293354 A1
(43) Pub. Date: Sep. 5, 2024

(54) METHODS AND COMPOSITIONS FOR TREATING OR PREVENTING A DISEASE OR A CONDITION

(71) Applicant: LIBI PHARM LTD, Rishon Lezion (IL)

(72) Inventors: Raziel RONEN, Hod Hasharon (IL); Dobroslav MELAMED, Rishon Lesion (IL)

(21) Appl. No.: 18/280,930

(22) PCT Filed: Mar. 25, 2022
(86) PCT No.: PCT/IL2022/050334

§ 371 Date: Sep. 7, 2023

Related U.S. Application Data
(60) Provisional application No. 63/165,793, filed on Mar. 25, 2021.

(51) Int. Cl. A61K 31/343; A61K 31/015; A61K 31/045; A61K 31/05; A61K 31/085; A61K 31/11; A61K 31/122; A61K 31/22; A61K 33/04; A61K 33/30; A61K 36/15; A61K 36/185; A61K 36/53; A61K 36/534; A61K 36/537; A61K 36/61; A61K 45/06; A61P 37/02

(52) U.S. Cl. CPC A61K 31/343; A61K 31/015; A61K 31/045; A61K 31/05; A61K 31/085; A61K 31/11; A61K 31/122; A61K 31/22; A61K 33/04; A61K 33/30; A61K 36/15; A61K 36/185; A61K 36/53; A61K 36/534; A61K 36/537; A61K 36/61; A61K 45/06; A61P 37/02

(57) ABSTRACT

The invention relates to a method and a composition for treating or preventing an inflammatory, autoimmune and/or Coronavirus disease and/or symptom. In some embodiments, the-invention relates to a method or a composition for treating or preventing a Coronavirus disease-19 (COVID-19) as well as preventing and curing long term COVID-19 side effects. The composition is a herbal mineral composition or includes the active ingredients that may be found in herbs together with a mineral.

### FIELD OF THE INVENTION

The invention relates to a method and a composition for treating or preventing an inflammatory, autoimmune and/or Coronavirus disease and/or symptom. In some embodiments, the invention relates to a method or a composition for treating or preventing a Coronavirus disease-19 (COVID-19) as well as preventing and curing long term COVID-19 side effects. The composition is a herbal mineral composition or includes the active ingredients that may be found in herbs together with a mineral.

### BACKGROUND OF THE INVENTION

Treating inflammation is of great importance in medicine. Existing treatments, however, are insufficient or have many side effects. Uncontrolled inflammation caused by either innate or adaptive immune responses, however, may lead to tissue damage and pain and is the underlying cause of many illnesses, including asthma, as well as other allergic, infectious, autoimmune, degenerative, and idiopathic diseases.

In view of the large number of types of inflammation and diseases associated with inflammation and the shortcomings of currently available drugs, there is a great need for new active agents to effectively treat these diseases and their symptoms without immunosuppressive adverse effects.

Inappropriate responses of the immune system may cause stressful symptoms to the organism. Exaggerated immune reactions to foreign substances or physical states which usually do not have a significant effect on the health of an animal or human may lead to allergies with symptoms ranging from mild reactions, such as skin irritations to life-threatening situations such as an anaphylactic shock or various types of vasculitis. Immune reactions to endogenous antigens may cause autoimmune disorders, such as, systemic lupus erythematosus (SLE), type for insulin dependent diabetes mellitus (TIDM or IDDM) and different forms of arthritis and the like. Immune responses occur in a coordinated manner, involving several cells and requiring communication by signaling molecules such as cytokines between the cells involved. Cytokines are secreted soluble proteins, peptides and glycoproteins acting as humoral regulators at nano-to picomolar concentrations behaving like classical hormones in that they act at a systemic level and which, either under normal or pathological conditions, modulate the functional activities of individual cells and tissues. Cytokines differ from hormones in that they are not produced by specialized cells organized in specialized glands, i.e. there is not a single organ or cellular source for these mediators as they are expressed by virtually all cells involved in innate and adaptive immunity such as epithelial cells, macrophages, dendritic cells (DC), natural killer (NK) cells and especially by T cells, prominent among which are T helper (Th) lymphocytes.

Depending on their respective functions, cytokines may be classified into three functional categories: regulating innate immune responses, regulating adaptive immune responses and stimulating hematopoiesis. Due to their pleiotropic activities within said three categories, e.g., concerning cell activation, proliferation, differentiation, recruitment, or other physiological responses, e.g., secretion of proteins characteristic for inflammation by target cells, disturbances of the cell signaling mediated by aberrantly regulated cytokine production have been found as a cause of many disorders associated with defective immune response, for example, inflammation and cancer.

Since December 2019, a novel coronavirus (nCOV) of animal origin began infecting humans, initiating a severe outbreak in China and a global pandemic. This virus, named “Severe Acute Respiratory Syndrome-related Coronavirus 2” (SARS-COV-2), can cause a severe and even fatal respiratory disease, called Coronavirus disease-19 (COVID-19), and leads to acute respiratory distress syndrome (ARDS) in some of the patients.

SARS-COV 2 Spike Protein (S) and Mechanism of Action: The SARS-CoV-2 spike protein (S) is the main molecule present at the surface of the virion. This large glycoprotein assembles in trimers that form a crown-like structure on the envelope. The spike protein is a multifunctional protein that contributes host receptor binding, cell tropism and pathogenesis.

A number of medicines (mostly antiviral drugs and vaccines) have been suggested as potential investigational therapies, many of which are now being or will soon be studied in clinical trials.

There is a need for a safe medicament, that can be administered orally that will be easy and fast to develop and manufacture and quickly available for treating the COVID 19 disease and/or inflammatory or autoimmune disease that will prevent, treat or reduce the cytokine storm in severe COVID 19 patients and further prevent late side effects of the disease and/or will be effective in treating or preventing inflammatory or autoimmune diseases or conditions.

### SUMMARY OF THE INVENTION

In some embodiments, there is provided a composition comprising one or more of the following ingredients:
1,8-Cineole
Citronellal;
Neral;
Geranial;
beta-Citronellol;
Estragole;
Menthol;
Cinnamicaldchyde;
Eugenol;
Linalyl acetate; and/or
Terpinene-4-ol.

In some embodiments, the composition further comprises
Camphene;
P-Menthone;
methyl-7-methylidene-1-propan-2-yl-2,3,4,4a,5,6-4;
hexahydro-1H-naphthalene;
(2R,5R)-5-methyl-2-(1-methylethyl)-Cyclohexanone;
alpha.-Humulene;
Delta.3-Carene;
Carene-;2
Thymol;
Para-Cymene;
Menthol;
Gamma-Terpinene;
Linalool; and/or
Delta-cadinene.

In some embodiments, the composition further comprises selenium and/or Zinc.

In some embodiments, the selenium and/or Zinc are treated by an acid.

In some embodiments, the ingredients are derived from one or more of Basil (Ócimum), Salvia Officinalis, Clove (Syzýgium aromáticum), Eucalyptus, Pinus maritima, Citronella, Mentha, Thyme or Tea tree.

In some embodiments, there is provided a method of treating ameliorating or preventing an inflammatory and/or autoimmune disease comprising the step of administering to the subject in need the mineral and herbal composition of the invention.

In some embodiments, there is provided a method of treating ameliorating or preventing a disease or a condition associated with increased levels of IL-6 in the blood comprising the step of administering to the subject in need the mineral and herbal composition of the invention.

In some embodiments, there is provided a method of treating ameliorating or preventing a corona disease comprising the step of administering to the subject in need the mineral and herbal composition of the invention.

In some embodiments, the corona disease is COVID 19 disease, HCoV-NL63, HCoV-OC43, HCOV-229E, HCoV-HKUI, SARS-COV (Severe Acute Respiratory Syndrome-Corona Virus), CoV MERS (Middle East Respiratory Syndrome virus or SARS-COV-2.

In some embodiments, the subject is with a severe COVID 19 disease.

In some embodiments, the subject has one or more of the following symptoms: a cytokine storm (aka cytokine release syndrome, or CRS), secondary hemophagocytic lymphohistiocytosis (sHLH), an acute respiratory distress syndrome (ARDS), unremitting fever, cytopenia, and hyperferritinaemia.

In some embodiments, the treatment ameliorates, cures, reduces or prevents at least one of the following symptoms: a cytokine storm (CCS), secondary hemophagocytic lymphohistiocytosis (sHLH), an acute respiratory distress syndrome (ARDS), unremitting fever, cytopenias, and hyperferritinaemia.

In some embodiments, the subject at risk of developing a severe COVID 19 disease and has the risk of developing one or more of the following symptoms: a cytokine storm (aka cytokine release syndrome, or CRS), secondary hemophagocytic lymphohistiocytosis (sHLH), an acute respiratory distress syndrome (ARDS), unremitting fever, cytopenia, or hyperferritinaemia.

In some embodiments, the treatment prevents at least one of the following symptoms: a cytokine storm, secondary hemophagocytic lymphohistiocytosis (sHLH), an acute respiratory distress syndrome (ARDS), unremitting fever, cytopenias, and hyperferritinaemia.

In some embodiments, the methods of the invention further comprising administrating an additional drug or therapy.

In some embodiments, the additional drug or therapy is an anti-cytokine therapy, anti-IL-6, anti-IL-1, an anti-viral drug, interferon-alpha2b, analgesic drug, corticosteroid or mechanical ventilation.

In some embodiments, the anti-viral drug is remdesivir, ribavirin, oseltamivir or zanamivir.

In some embodiments, the treatment is associated with reduction of one or more of IL-6, TNFα, IL-2, IL-1beta or IL8.

In some embodiments, the subject has elevated ferritin or IL-6, TNFα, IL-2, IL-1beta or IL8 in the blood.

In some embodiments, the subject has elevated increased interleukin (IL)-2, IL-3, granulocyte-colony stimulating factor (GCSF), and other pro-inflammatory cytokines.

In some embodiments, the composition is administered by orally.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-1J shows inhibition of the activation of bronchial epithelial cells (BEAS-2B) and lung fibroblasts (MRC-5 cells) activation by ImmuneCov. ImmuneCov inhibited the synthesis and release of IL-1beta (p<0.001), IL-6 (p<0.05), IL-8 (p<0.05) and TNF-alpha (p<0.001) in BEAS-2B (FIGS. 1A, 1B, 1C and 1D, respectively), and reduced the synthesis and release of IL-1beta (p<0.001), IL-6 (p<0.001) and TNF-alpha (p<0.001) in MRC-5 cells (FIGS. 1E, IF, 1G and 1H) . ***p<0.001; **p<0.01 and *p<0.05. Co=control group (only the cells); IC=ImmuneCov group (cells+ImmuneCov); SarsCov2=Infected group (cells incubated with blood of patient infected with COVID-19) and SarsCov2+IC=Infected group treated with ImmuneCov.

FIG. 2 shows activation of the synethsis and/or release of IL-1RA in bronchial epithelial cells (BEAS-2B) by ImmuneCov. The experiment was designed as described in Example 1 and FIG. 1A-H. Co=control group (only the cells); IC=ImmuneCov group (cells+ImmuneCov); SarsCov2=Infected group (cells incubated with blood of patient infected with COVID-19) and SarsCov2+IC=Infected group treated with ImmuneCov.

FIG. 3-Thorax CT before and after ImmuneCov for 12 days: In panel A (two upper panels) the images demonstrate approximately 50%-75% of ground-glass opacities in the whole lung parenchyma, while in panel B (two lower panels) after 12 days of treatment, the images demonstrate approximately 25%-50% of ground-glass opacities in the whole lung parenchyma.

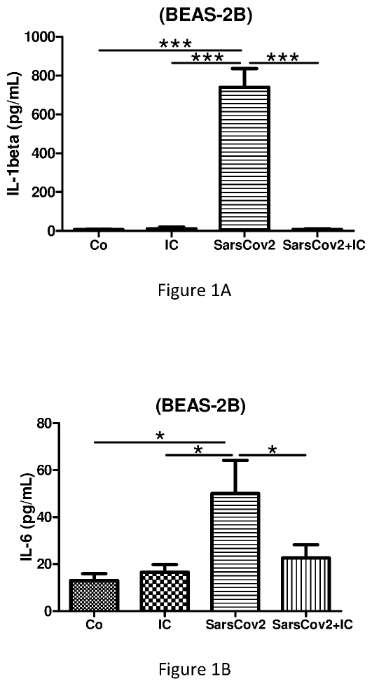

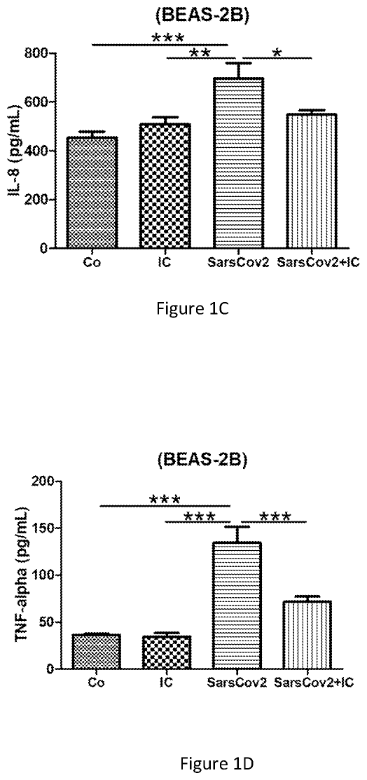

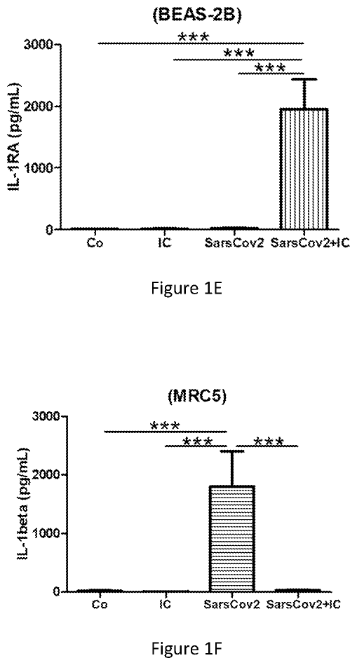

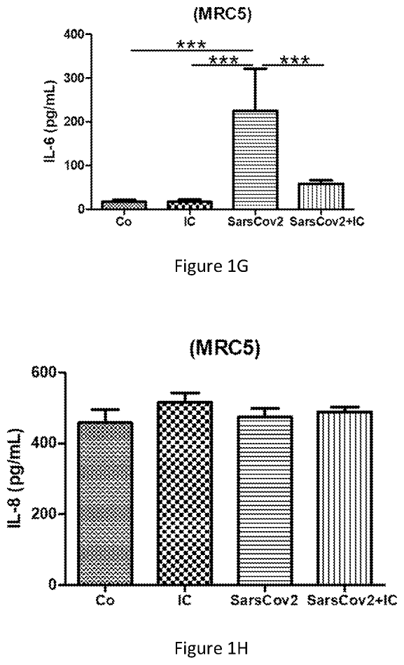

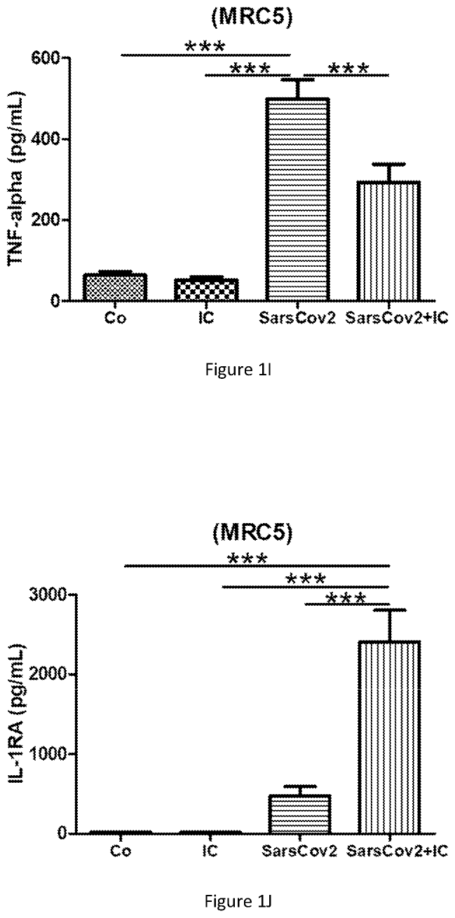

### DETAILED EMBODIMENTS OF THE INVENTION

In some embodiments, there is provided a herbal-mineral composition or a composition comprising the active ingredients from herbs together with a mineral comprising one or more, two or more, three or more, four or more, five or more, six or more, seven or more, eight or more or all of the following:
1,8-Cineole
Citronellal
Neral
Geranial
beta-Citronellol
Estragole
Menthol
Cinnamicaldchyde
Eugenol
Linalyl acetate
Terpinene-4-ol

In some embodiments, the amount of each of these ingredients is between 0.01% to 10% w/w. In some embodiments, the amount of each of these ingredients is between 0.1% to 10% w/w. In some embodiments, the amount of each of these ingredients is between 0.2% to 8% w/w. In some embodiments, the amount of each of these ingredients is between 0.4% to 6% w/w. In some embodiments, the amount of each of these ingredients is between 0.5% to 5% w/w. In some embodiments, the amount of each of these ingredients is between 0.7% to 3% w/w. In some embodiments, the amount of each of these ingredients is between 1% to 3% w/w. In some embodiments, the amount of each of these ingredients around 1% w/w. In some embodiments, the amount of each of these ingredients around 1% w/w and the amount of Estragole is between 2-3%. In some embodiments, the amount of each of these ingredients around 1% w/w and the amount of Estragole is about 2.4%.

In some embodiments, the composition further the following two or more, three or more, four or more, five or more, six or more, seven or more, eight or more or all of the following:
Benzeneethanol
(1R)-2,6,6-Trimethylbicyclo[3.1.1]hept-2-ene
Camphene
P-Menthone
methyl-7-methylidene-1-propan-2-yl-2,3,4,4a,5,6-4
hexahydro-1H-naphthalene
(2R,5R)-5-methyl-2-(1-methylethyl)-Cyclohexanone
alpha.-Humulene
Delta.3-Carene
Carene-2
Thymol
Para-Cymene
Menthol
Gamma-Terpinene
Linalool
Delta-cadinene

In some embodiments, the amount of each of these ingredients is between 0.005% to 5% w/w. In some embodiments, the amount of each of these ingredients is between 0.05% to 5% w/w. In some embodiments, the amount of each of these ingredients is between 0.1% to 4% w/w. In some embodiments, the amount of each of these ingredients is between 0.2% to 3% w/w. In some embodiments, the amount of each of these ingredients is between 0.25% to 3% w/w. In some embodiments, the amount of each of these ingredients is between 0.35% to 2% w/w. In some embodiments, the amount of each of these ingredients is between 0.5% to 2% w/w. In some embodiments, the amount of each of these ingredients around 0.5% w/w.

In some embodiments, these active ingredients are derived from alcoholic and lipid extracts of one or more, two or more, three or more, four or more, five or more, six or more, seven or more or all of the following:
Basil (Ócimum)
Salvia Officinalis
Clove (Syzýgium aromáticum)
Eucalyptus
Pinus maritima
Citronella
Mentha
Thyme
Tea tree

In some embodiments, the alcohol is ethyl alcohol.

In some embodiments, the composition further comprises selenium (SE) and or Zinc (Zn). In some embodiments, the amount of the Se and or the Zn is 0.001%-5% W/W.

In some embodiments of the invention, the method for manufacturing the composition is as follows: add 5%-19% w/w herbs containing the organic molecules described above into ethanol 70-80% v/v at a temperature of between 20-30° C., for 2-60 days. Grinding for between one to 100 minutes, heating to 30-50° C. and filtering. Testing the filtrate for the organic molecules. Adding the minerals.

In some embodiments, the minerals are prepared as follows: Minerals are treated with H₂SO₄ in a liquid form for between one minute to 50 hours to enable superior bioavailability and absorption. The ration of the mineral acid is 1-50% mineral in 50-99% acid. The mixture is than filtered in polyethylene filter and dired by using vaccum or heat.

It is noted that the same composition but without the addition of minerals i.e. Se and/or Zn was tested in both human and animals but did not show any effects on viruses (data not shown).

In some embodiments, the composition of the invention causes inhibition of the synthesis and or the release of IL-1beta IL-6, IL-8 and TNF-alpha in the blood of a subject in need.

In some embodiments, the composition of the invention causes of the synthesis and/or the release of IL-1Ra in the blood of a subject in need and accordingly, has an unexpected effect beneficial effect in inflammatory diseases.

In some embodiments of the invention, there is provided a method of treating ameliorating or preventing an inflammatory and/or autoimmune disease comprising the step of administering to the subject in need a therapeutically effective amount of the mineral and herbal composition of the invention.

In some embodiments, the inflammatory and/or autoimmune disease is one or more of systemic lupus erythematosus (SLE), various forms of arthritis including but not limited to rheumatoid arthritis (RA), type 1 or insulin dependent diabetes mellitus (TIDM or IDDM), Sjögren's syndrome, Dermatomyositis, Multiple Sclerosis (MS), psoriasis, chronic psoriasis, myositis, systemic scleroderma, autoimmune thyroiditis and cancer including leukemia.

In some embodiments, the disease is selected from the group consisting of: acute pain, chronic pain, pruritus, hyponatremia, edema, ileus, tussis, glaucoma, and an inflammatory disease, wherein an acute inflammatory disease or a the inflammatory disease is chronic inflammatory disease.

In some embodiments, the inflammatory disease is selected from the group consisting of: cardiovascular inflammation, neurological inflammation, skeletal inflammation, skin inflammation, muscular inflammation, gastrointestinal inflammation, ocular inflammation, otic inflammation, inflammation due to insect bites, inflammation due to wound healing, atherosclerosis, ischemia, restenosis, vasculitis, asthma, Sjogren's syndrome, pulmonary inflammation, chronic airway inflammation, chronic obstructive pulmonary disease (COPD), allergy, psoriasis, psoriatic arthritis, eczema, scleroderma, atopic dermatitis, systemic lupus erythematosus, arthritis, synovitis, osteomyelitis, rheumatoid arthritis, osteoarthritis, ankylosing spondylitis; septicemia, septic shock, diabetes, glucose intolerance, insulin resistance, obesity, colitis, ulcerative colitis, Crohn's disease, IBD, IBS, inflammatory diseases and conditions due to tumor proliferation, inflammatory diseases and conditions due to tumor metastasis, and inflammatory diseases and conditions due to transplantation rejection.

In some embodiments, the inflammatory disease is selected from the group consisting of: sepsis, an inflammatory disease of the joints, vulvovaginitis, an inflammatory disease of the brain, an inflammatory disease of the skin, an inflammatory disease of the hair follicle, an inflammatory disease of the urogenital tract, an inflammatory disease of the eyes, sinusitis, tenosynovitis, bursitis, tendonitis, lateral epicondylitis, adhesive capsulitis, autoimmune inflammation, contact dermatitis, atopic eczema, alopecia areata, scleroderma, a fibrotic disease, urticaria, lichen planus, lymphoma, and a disease characterized by mast cell involvements.

In some embodiments, the inflammatory disease is an inflammatory disease of the gastrointestinal tract.

In some embodiments, the inflammatory disease of the gastrointestinal tract is an inflammatory bowel disease. In some embodiments, the inflammatory bowel disease is selected from the group consisting of: Crohn's disease colitis ulcerosa. In some embodiments, the inflammatory disease of the gastrointestinal tract is selected from the group consisting of: inflammation of the gall bladder, inflammatory pseudopolyps, colitis cystica profunda, pneumatosis cystoides intestinales, pancreatitis, and appendicitis.

In some embodiments, there is provided a method of treating ameliorating or preventing a disease or a condition associated with increased levels of IL-6 in the blood comprising the step of administering to the subject in need a therapeutically effective amount of the mineral and herbal composition of the invention. IL-6 levels are rapidly elevated in the circulation in numerous infectious, inflammatory, autoimmune diseases, and in some cancers, in association with increased synthesis of other cytokines, stimulated by infection, trauma, and immunological challenge. IL-6 has been implicated in various diseases and disorders, including multiple myeloma, Bone Marrow Transplantation, lymphomas, neurological disorders, such as neurodegeneration, astrocytosis, and cerebral angiogenesis, autoimmune disorders (e.g., rheumatoid arthritis), inflammatory diseases, Alzheimer's disease, myocardial infarction, Paget's disease, osteoporosis, solid tumors, prostate and bladder cancers, septic shock, transplants, acute infections of the central nervous system, cardiac myxoma, tumor-induced cachexia, cancer-associated depression, and cerebral edema secondary to brain tumors. Generally, it is known that IL-6 is abnormally produced in some inflammatory, autoimmune, and neoplasmic diseases. It has been proposed that abnormal production of IL-6 is an aspect of the mechanisms of these diseases. In particular, it is known that IL-6 is associated with neuropathological processes, and its level in blood is increased in diseases invading the central nervous system. It has been found that IL-6 increases the level of tau epitope, by stimulating the dementia-associated phosphorylation of the tau protein in neuronal cells. Mice lacking IL-6 have enhanced resistance to glutamate toxicity and increased viability of neuronal cells. It has also been reported that the abnormal expression of IL-6 is a pathogenic mechanism in other diseases, including cardiac myxoma, uterine cancer, multiple myeloma, histiocytomas), allergic diseases, rheumatoid arthritis, diabetes, multiple sclerosis, Systemic Lupus Erythematosus, septic shock, bacterial infections, viral infections, osteoporosis, chronic immunodeficiency syndrome and autoimmune immunodeficiency syndromes, including AIDS and inflammatory diseases, including inflammatory bowel diseases (such as Crohn's disease and ulcerative colitis). Interleukin-6 is secreted by many advanced cancers, such as hormone-independent prostate cancer, and is believed to be a growth factor for such cancers. Additionally, the secretion of IL-6 by cancer cells is believed to cause cachexia, the wasting syndrome characteristic of advanced cancers. Thus, reducing the level of IL-6 would be useful in treating such cancers. IL-6 also plays a key role in B cell development. Autoimmune diseases with a significant antibody component, such as rheumatoid arthritis, could be treated by decreasing IL-6 levels. Disorders involving B cell proliferation, such as multiple myeloma and B cell lymphoma, could also be treated by reducing IL-6 activity. Additionally, IL-6 plays an important role in bone remodeling by promoting bone resorption. Reducing IL-6 activity would have the effect of reducing bone resorption and could be used to treat osteoporosis.

Accordingly, in an embodiment of the invention, there is provided a composition that reduces the levels of IL-6, which are believed to be associated with the pathogenic mechanisms of these various diseases and conditions.

In an embodiment of the invention, there is provided a method of treating ameliorating or preventing a corona disease comprising the step of administering to the subject in need the mineral and herbal composition of the invention.

In some embodiments, the corona disease is COVID 19 disease, HCoV-NL63, HCoV-OC43, HCOV-229E, HCoV-HKUI, SARS-COV (Severe Acute Respiratory Syndrome-Corona Virus), CoV MERS (Middle East Respiratory Syndrome virus or SARS-COV-2.

In some embodiments, the subject is with a severe COVID 19 disease.

In some embodiments, the subject has one or more of the following symptoms: a cytokine storm (aka cytokine release syndrome, or CRS), secondary hemophagocytic lymphohistiocytosis (sHLH), an acute respiratory distress syndrome (ARDS), unremitting fever, cytopenia, and hyperferritinaemia.

In some embodiments, the treatment ameliorates, cures, reduces or prevents at least one of the following symptoms: a cytokine storm (CCS), secondary hemophagocytic lymphohistiocytosis (sHLH), an acute respiratory distress syndrome (ARDS), unremitting fever, cytopenias, and hyperferritinaemia.

In some embodiments, the subject at risk of developing a severe COVID 19 disease and has the risk of developing one or more of the following symptoms: a cytokine storm (aka cytokine release syndrome, or CRS), secondary hemophagocytic lymphohistiocytosis (sHLH), an acute respiratory distress syndrome (ARDS), unremitting fever, cytopenia, or hyperferritinaemia.

In some embodiments, the treatment prevents at least one of the following symptoms: a cytokine storm, secondary hemophagocytic lymphohistiocytosis (sHLH), an acute respiratory distress syndrome (ARDS), unremitting fever, cytopenias, and hyperferritinaemia.

In some embodiments, there is provided a method of treating ameliorating, curing, reducing or preventing lung or liver fibrosis.

In some embodiments, the methods of the invention further comprising administrating an additional drug or therapy.

In some embodiments, the additional drug or therapy is an anti-cytokine therapy, anti-IL-6, anti-IL-1, an anti-viral drug, interferon-alpha2b, analgesic drug, corticosteroid or mechanical ventilation.

In some embodiments, the anti-viral drug is remdesivir, ribavirin, oseltamivir or zanamivir.

In some embodiments, the treatment is associated with reduction of one or more of IL-6, TNFα, IL-2, IL-1beta or IL8.

In some embodiments, the subject has elevated ferritin or IL-6, TNFα, IL-2, IL-1beta or IL8 in the blood.

In some embodiments, the subject has elevated increased interleukin (IL)-2, IL-3, granulocyte-colony stimulating factor (GCSF), and other pro-inflammatory cytokines.

In some embodiments, the composition is administered by orally.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the terms “treatment,” “treat,” and “treating” refer to reversing, alleviating, delaying the onset of, or inhibiting the progress of a disease or disorder, or one or more symptoms thereof, as described herein. In some embodiments, the composition of the invention inhibits or prevents the post COVID 19 effects. In some embodiments, treatment may be administered after one or more symptoms have developed. In other embodiments, treatment may be administered in the absence of symptoms. For example, treatment may be administered to a susceptible individual, e.g. subject that is in risk of developing severe COVID 19 disease prior to the onset of symptoms (e.g., in light of a history of symptoms, the risk factors, such as age or respiratory or cardiac diseases and/or in light of genetic or other susceptibility factors). Treatment may also be continued after symptoms have resolved, for example to prevent or delay their recurrence. The term also includes positive prognosis of disease, i.e., the subject may have reduced or no viral titers upon administration of a therapeutic agent.

In some embodiments, the term includes treatment of one or more of the following symptoms in a patient infected with severe COVID 19: a cytokine storm (aka cytokine release syndrome, or CRS), secondary hemophagocytic lymphohistiocytosis (sHLH), an acute respiratory distress syndrome (ARDS). unremitting fever, cytopenias, and hyperferritinaemia.

Severe COVID 19 subjects or subjects at risk of developing severe COVID 19 subjects can be diagnosed as follows: Tests to diagnose cytokine storms are available in hospitals. An elevation in the serum ferritin can be detected. Typically, the value is less than 200 nanograms per ML and for COVID-19 related cytokine storms, it tends to run between 500 to the low thousands. Further, COVID-19 severe patients, for whatever reason, seem to be very lymphopenic, meaning their lymphocyte count tends to be less than a thousand per microliter.

Cytokine storms also can be indicated by liver enzyme abnormalities. COVID 19 severe subjects tend to have high D dimers and lactate dehydrogenase (LDH) levels also tend to go up as well.

The terms “prevent”, “preventing” or “prevention” refer to inhibition of manifestation of COVID 19 infection or any symptoms or indications of COVID 19 infection upon administration of the compositions of the invention. The term includes prevention of spread of infection in a subject exposed to the virus or at risk of having COVID 19 infection. In some embodiments, the term includes prevention of one or more of the following symptoms: a cytokine storm (aka cytokine release syndrome, or CRS), secondary hemophagocytic lymphohistiocytosis (sHLH), an acute respiratory distress syndrome (ARDS). unremitting fever, cytopenias, and hyperferritinaemia. In some embodiments, the term refers to side effects that appear after recovery from COVID 19 infection.

The term “subject” as used herein refers to a mammal. A subject therefore refers to, for example, dogs, cats, horses, cows, pigs, guinea pigs, and the like. Preferably the subject is a human. When the subject is a human, the subject may be either a patient or a healthy human.

In some embodiments, the therapeutically effective amount of a composition as defined herein, can be administered to a subject alone or admixed with a pharmaceutically acceptable carrier.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Pharmaceutically acceptable carrier also refers to a non-toxic solid, semisolid or liquid filler, diluent, encapsulating material or formulation auxiliary of any.

Compositions described herein may be administered orally, parenterally, by inhalation spray, dry powder inhalation, topically, rectally, nasally, buccally, or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the subject in need may inhale vapor from an electronic cigarette or similar device which is similar to a conventional cigarette.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

### Definitions

As used herein, the singular forms “a”, “an” and “the” are intended to include the plural forms as well, unless the context clearly indicates otherwise. To the extent that the terms “including”, “includes”, “having”, “has”, “with”, or variants thereof are used in either the description and/or the claims, such terms are intended to be inclusive in a manner similar to the term “comprising.”

The term “about” or “approximately” means within an acceptable error range for the particular value as determined by a person skilled in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

### EXAMPLE 1

In examples 1 and 2, the composition (termed here ImmuneCov) was as follows:

### Group 1

1,8-Cineole
Citronellal
Neral
Geranial
beta-Citronellol
Estragole
Menthol
Cinnamicaldchyde
Eugenol
Linalyl acetate
Terpinene-4-ol

The amount of each of the above ingredients of group 1 was 1% w/w.

### Group 2

Benzeneethanol
(1R)-2,6,6-Trimethylbicyclo[3.1.1]hept-2-ene
Camphene
P-Menthone
methyl-7-methylidene-1-propan-2-yl-2,3,4,4a,5,6-4
hexahydro-1H-naphthalene
(2R,5R)-5-methyl-2-(1-methylethyl)-Cyclohexanone
alpha.-Humulene
Delta.3-Carene
Carene-2
Thymol
Para-Cymene
Menthol
Gamma-Terpinene
Linalool
Delta-cadinene

The amount of each of the above ingredients of group 2 was 0.5% w/w.

### Group 3

Selenium
Zinc

The amount of each of the above ingredients of group 3 was 0.25% w/w.

It is noted that the product has also other names, such as, DSM-IMMUNE CARE, SELELIFE IMMUNE CARE, IMMUNE COV, VIRLAZA, IMMUNO COV

### In Vitro Studies in Cell Lines with COVID-19 and the Effect of ImmuneCov (the Formulation of the Invention) on Different Pro-Inflammatory Cytokines

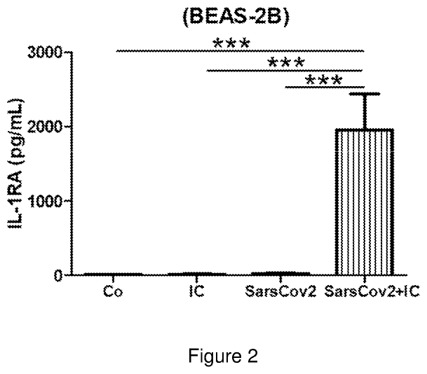

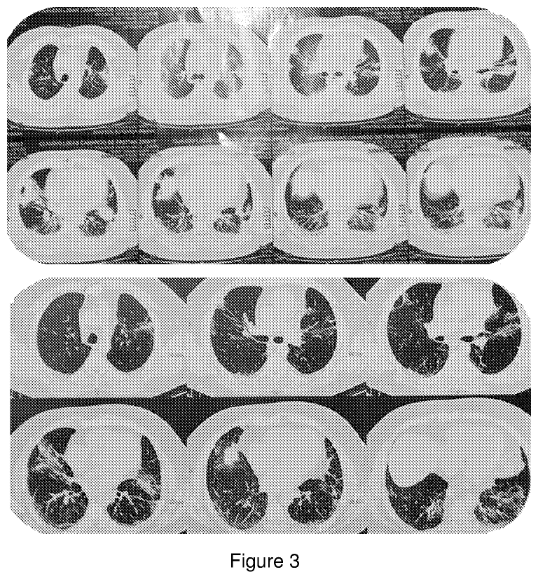

The experiments were done using two cells lines, which have a central role in the physiopathology of COVID-19, BEAS-2B cells (bronchial epithelial cells) and MRC-5 cells (lung fibroblast cells), which were stimulated by whole blood cells from COVID-19 patient with viral load ranging CT 15-18, obtained from acute phase of disease (maximum of 24 hours of hospitalization). 5×10⁴ BEAS-2B cells and 1×10⁴ MRC-5 cells were cultivated in 2 ml of fresh whole blood of COVID-19 patient, for an initial incubation with 50 ul of ImmuneCov (the formulation of the invention) for 1 hour, followed by an additional period of incubation of 3 hours, into a CO₂ incubator, with 5% CO₂ at 37° C. degrees. After 4 hours of incubation, the cells were harvested and centrifuged at 900 g at 4° C. degrees, for 7 minutes. The supernatant was used for measurement of pro-inflammatory cytokines (IL-1beta, IL-6, IL-8 and TNF-alpha), which are key cytokines for COVID-19 physiopathology, progression and severity, as well as of anti-inflammatory cytokines (IL-1RA and IL-10). The results demonstrated that ImmuneCov significantly inhibited the activation of bronchial epithelial cells (BEAS-2B), as it happens in the real-life in vivo context during COVID-19 infection, since ImmuneCov inhibited the synthesis and release of IL-1beta (p<0.001), IL-6 (p<0.05), IL-8 (p<0.05) and TNF-alpha (p<0.001). Of note, according to the protocol used, adding ImmuneCov only after 1 hour incubation of BEAS-2B cells with infected blood from COVID-19 patient, strongly suggests that ImmuneCov could be successfully used in the early phase for COVID-19 patients, to avoid the aggravation of the disease, which happens due to intense synthesis and release of pro-inflammatory cytokines by bronchial epithelial cells. In terms of possible immunological mechanism underlying such effects, the present study revealed that ImmuneCov induced synthesis and release of IL-1RA in BEAS-2B cells, which is a potent endogenous anti-inflammatory cytokine. In addition, following this initial pro-inflammatory response, an activation of lung fibroblasts is seemed in COVID-19 patients, which leads to lung fibrosis, compromise the lung function, the quality of life and the general health status of such patients for a long period. In this case, the present study demonstrated that ImmuneCov efficiently reduced lung fibroblasts (MRC-5 cells) activation, since ImmuneCov reduced the synthesis and release of IL-1beta (p<0.001), IL-6 (p<0.001) and TNF-alpha (p<0.001), which are classical cytokines used as biomarker of fibroblast activation. Importantly, the levels of IL-8 were not changed in MRC-5 after stimulation with infected blood of COVID-19 patients and also not changed after ImmuneCov stimulation. In this sense, is plausible to hypothesize that ImmuneCov, also could inhibit the lung fibrosis observed in more severe cases of COVID-19. Again, concerning the possible anti-inflammatory immunological mechanism involved in the effects of ImmuneCov, its was able to induce the synthesis and release of the anti-inflammatory cytokine IL-IRA, which is the trigger anti-inflammatory cytokine suggesting deactivation of lung fibroblasts, induced by COVID-19. So, with these consistent results, we are able to prove a potent anti-inflammatory and anti-fibrotic effect of ImmuneCov in the context of COVID-19, open a new opportunity for early intervention in individuals with COVID-19. ***p<0.001; **p<0.01 and *p<0.05. Co=control group (only the cells); IC=ImmuneCov group (cells+ImmuneCov); SarsCov2=Infected group (cells incubated with blood of patient infected with COVID-19) and SarsCov2+IC=Infected group treated with ImmuneCov. It is noted that the levels of IL-10 (an immunomodulatory and sometimes anti-inflammatory cytokine) were also measured, but no differences among all groups were found. Lastly, as can be seen in FIG. 2, the levels of IL1Ra in BEAS-2B cells (bronchial epithelial cells) were highly elevated showing that Virlaza/ImmuneCov activating anti inflammatorily mechanism, please complete what is the meaning of that.

### EXAMPLE 2

### ImmuneCov Improves Cardiopulmonary Recovery of Post-Covid-19 Patient: A Case Report

A 50-year-old man, without any history of previous respiratory diseases, with historic of two cardiovascular surgeries (mamarian and safena), presenting grade II obesity (BMI 35.91), flu-like symptoms, fatigue and breathlessness, partial oxygen saturation (SpO2 %) of 92%, laboratory diagnosis and computerized tomography (CT) of the lungs (>75% ground-glass opacities) characterizing acute respiratory distress syndrome (ARDS), who was additionally diagnosed with coronavirus disease 19 (COVID-19) by RT-PCR. After twelve days of hospitalization, under treatment with antibiotic (Amoxicillin 500 mg/2×/day and Clavulanate Potassium 125 mg/2×/day), corticosteroid (Prednisone 20 mg/day) and sodium enoxaparin (100 mg/day), the patient has been discharged from the hospital, with a new CT presenting 50% of ground-glass opacities of the lungs. The main complaints of the patient after hospital discharge were acute fatigue for minimal effort, after standing of a chair, for example. So, the following evaluations were performed before and after 12 days of use of ImmuneCov (20 drops, 2×/day): the SpO2 % was evaluated at rest, the lung function by spirometry, the levels of exhaled nitric oxide, the maximal inspiratory (MIP) and expiratory (MEP), and the hand grip strength. The results demonstrated that 12 days of ImmuneCov resulted in improvement of: lung function, notably on forced vital capacity (FVC; 1.91 L×2.31 L; 41.2%×61.7% of predicted), forced expiratory volume in the first second (VEF1; 1.51 L×1.68 L; 40.4%×55.9% of predicted), inspiratory vital capacity (VC In; 1.59 L×1.63 L, 36.3%×46.7% of predicted), forced expiratory flow 25% (FEF 25%; 0.47 L/s×0.58 L/s, 35.8%×58.3% of predicted), forced expiratory flow 50% (FEF 50%; 1.42 L/s×1.62 L/s, 37.4%×41.9% of predicted), forced expiratory flow 75% (FEF 75%; 2.91 L/s×3.58 L/s, 48.3%×49.7% of predicted), peak expiratory flow (PEF; 2.99 L/s×3.97 L/s, 36.4%×38%). Importantly, the CT revealed a significant reduction in ground-glass opacities, reducing from >75% to 25-50% after 12 days of ImmuneCov (see in FIG. 3). The levels of fractional exhaled nitric oxide (FeNO) which reflects the pulmonary inflammation, was initially of 28 parts per billion (ppb) to 11 ppb after treatment with ImmuneCov. SpO2 % also presented very significant recovery, once SpO2 of 92% was initially measured after hospital discharge reaching a SpO2 of 99% after 12 days of treatment with ImmuneCov. The strength of respiratory muscles also presented improvement after treatment with ImmuneCov, where MIP was −100 cmH₂O×120 cmH₂O and MEP 40 cmH₂O×60 cmH₂O. Concerning the peripheral muscle strength measured by hand grip strength, the results demonstrated that ImmuneCov improved hand grip strength on right (28.9 Kg×36.2 Kg) and left (19.8 Kg×23.1 Kg) hand. Therefore, the present case study reveals beneficial effects of ImmuneCov on clinical and functional parameters of severe post-COVID-19 patient, significantly improving their recovery.

1. A composition comprising one or more of the following ingredients:
1,8-Cineole
Citronellal;
Neral;
Geranial;
beta-Citronellol;
Estragole;
Menthol;
Cinnamicaldchyde;
Eugenol;
Linalyl acetate; and/or Terpinene-4-ol.

2. The composition of claim 1, wherein the composition further comprises
(1R)-2,6,6-Trimethylbicyclo[3.1.1]hept-2-ene;
Camphene;
P-Menthone;
methyl-7-methylidene-1-propan-2-yl-2,3,4,4a,5,6-4;
hexahydro-1H-naphthalene;
(2R,5R)-5-methyl-2-(1-methylethyl)-Cyclohexanone;
alpha.-Humulene;
Delta.3-Carene;
Carene-;2
Thymol;
Para-Cymene;
Menthol;
Gamma-Terpinene;
Linalool; and/or
Delta-cadinene.

3. The composition of claim 1 further comprises selenium and/or Zinc.

4. The composition of claim 3, wherein the selenium and/or Zinc are treated by an acid.

5. The composition of claim 1, wherein the ingredients are derived from one or more of Basil (Ócimum), Salvia Officinalis, Clove (Syzýgium aromáticum), Eucalyptus, Pinus maritima, Citronella, Mentha, Thyme or Tea tree.

6. A method of treating ameliorating or preventing an inflammatory and/or autoimmune disease comprising the step of administering to the subject in need the mineral and herbal composition according to claim 1.

7. A method of treating ameliorating or preventing a disease or a condition associated with increased levels of IL-6 in the blood comprising the step of administering to the subject in need the mineral and herbal composition according to claim 1.

8. A method of treating ameliorating or preventing a corona disease comprising the step of administering to the subject in need the mineral and herbal composition according to claim 1.

9. The method of claim 8, wherein the corona disease is COVID 19 disease, HCoV-NL63, HCoV-OC43, HCOV-229E, HCoV-HKUI, SARS-COV (Severe Acute Respiratory Syndrome-Corona Virus), CoV MERS (Middle East Respiratory Syndrome virus or SARS-COV-2.

10. The method of claim 8, wherein the subject is with a severe COVID 19 disease.

11. The method of claim 8, wherein the subject has one or more of the following symptoms: a cytokine storm (aka cytokine release syndrome, or CRS), secondary hemophagocytic lymphohistiocytosis (sHLH), an acute respiratory distress syndrome (ARDS), unremitting fever, cytopenia, and hyperferritinaemia.

12. The method of claim 8, wherein the treatment ameliorates, cures, reduces or prevents at least one of the following symptoms: a cytokine storm (CCS), secondary hemophagocytic lymphohistiocytosis (sHLH), an acute respiratory distress syndrome (ARDS), unremitting fever, cytopenias, and hyperferritinaemia.

13. The method of claim 8, wherein the subject at risk of developing a severe COVID 19 disease and has the risk of developing one or more of the following symptoms: a cytokine storm (aka cytokine release syndrome, or CRS), secondary hemophagocytic lymphohistiocytosis (sHLH), an acute respiratory distress syndrome (ARDS), unremitting fever, cytopenia, or hyperferritinaemia.

14. The method of claim 13, wherein the treatment prevents at least one of the following symptoms: a cytokine storm, secondary hemophagocytic lymphohistiocytosis (sHLH), an acute respiratory distress syndrome (ARDS), unremitting fever, cytopenias, and hyperferritinaemia.

15. The method of claim 6, wherein the method further comprising administrating an additional drug or therapy.

16. The method of claim 15, wherein the additional drug or therapy is an anti-cytokine therapy, anti-IL-6, anti-IL-1, an anti-viral drug, interferon-alpha2b, analgesic drug, corticosteroid or mechanical ventilation.

17. The method of claim 16, wherein the anti-viral drug is remdesivir, ribavirin, oseltamivir or zanamivir.

18. The method of claim 6, wherein the treatment is associated with reduction of one or more of IL-6, TNFα, IL-2, IL-1beta or IL8.

19. The method of claim 6, wherein the subject has elevated ferritin or IL-6, TNFα, IL-2, IL-1beta or IL8 in the blood.

20. The method of claim 6, wherein the subject has elevated increased interleukin (IL)-2, IL-3, granulocyte-colony stimulating factor (GCSF), and other pro-inflammatory cytokines.

21. The method of claim 6, wherein the composition is administered by orally.
